(19) United States
(12) Patent Application Publication
(10) Pub. No.: US 2018/0080915 A1
(43) Pub. Date: Mar. 22, 2018

(54) METHODS FOR QUANTIFYING OLEFINS IN HYDROCARBONS

(71) Applicant: ExxonMobil Research and Engineering Company, Annandale, NJ (US)

(72) Inventors: Birbal Chawla, Sterling, VA (US); Cathleen Yung, Beaumont, TX (US); Bryan E. Hagee, Hamilton, NJ (US)

(21) Appl. No.: 15/696,628

(22) Filed: Sep. 6, 2017

Related U.S. Application Data
(60) Provisional application No. 62/396,514, filed on Sep. 19, 2016.

(51) Int. Cl. G01N 33/28; G01N 1/34; C07C 7/13; C07C 7/00; C07C 7/11; G01N 24/08

(52) U.S. Cl. CPC G01N 33/28; G01N 1/34; C07C 7/13; G01N 1/405; C07C 7/11; G01N 24/082; C07C 7/005

(57) ABSTRACT

Methods and systems for quantifying olefinic hydrocarbons in a hydrocarbon sample are provided. The methods can include separating olefinic hydrocarbons from the hydrocarbon sample by high pressure liquid chromatography. The methods can further include measuring the proton resonance signals of the separated olefinic hydrocarbons and quantifying the weight percentage of different subtypes of olefinic hydrocarbons and hence the weight percentage of total olefinic hydrocarbons in the hydrocarbon sample based at least in part on the proton resonance signals.

### CROSS-REFERENCE TO RELATED APPLICATIONS

This application claims priority to U.S. Provisional Application Ser. No. 62/396,514 filed Sep. 19, 2016, which is herein incorporated by reference in its entirety.

### BACKGROUND

### Field of the Disclosed Subject Matter

The present disclosed subject matter relates to methods and systems for quantification and/or characterization of olefins in hydrocarbon samples. In certain exemplary embodiments, the methods comprise chromatographic separation of the olefins in the sample and analysis of the separated olefins by proton nuclear magnetic resonance.

### Description of Related Art

Olefins can be associated with diminished performance of finished hydrocarbons. For example, the presence of olefins in a lubricant basestock is believed to be associated with impaired corrosion and oxidation prevention. It is therefore useful to quantify and characterize the olefinic content of hydrocarbon basestocks, particularly as a wider variety of feedstocks are considered for refinement into finished lubricants.

Conventional methodologies for the measurement of olefin content of basestocks and vacuum gas oils are not sufficiently sensitive or specific for all commercial applications. For example, ASTM D1159 (“Standard Test Method for Bromine Numbers of Petroleum Distillates and Commercial Aliphatic Olefins by Electrometric Titration”) is not specific for olefins, but rather measures all molecules that react with the bromine reagent. Nuclear magnetic resonance (NMR) spectroscopy can specifically detect olefinic compounds in hydrocarbon samples, but are limited in sensitivity by the detection limits of NMR spectroscopy. Commercially available testing methods for olefins in petroleum streams, such as offered by Maxxam Analytics (“Test Method for Determination of Olefin Content of Crude Oils, Condensates, and Diluents by 1H NMR”) do not distinguish among the different types of olefins that can be measured by NMR spectroscopy.

As such, there remains a need for sufficiently sensitive and specific methods and systems to measure a relatively low level of olefins in hydrocarbon basestocks.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, a method for quantifying olefins in a hydrocarbon sample includes providing a hydrocarbon sample containing olefinic hydrocarbons, separating substantially all olefinic hydrocarbons from the sample into an olefinic fraction, spectroscopically measuring proton resonance signals of the olefinic fraction, and quantifying the olefinic hydrocarbons based at least in part on the proton resonance signals of the olefinic fraction.

As embodied herein, separating olefinic hydrocarbons from a sample into an olefinic fraction can be performed with a high-pressure liquid chromatography (HPLC) apparatus, and can include contacting the sample with a substrate exhibiting preferential affinity for olefinic hydrocarbons to immobilize the olefinic hydrocarbons on the substrate and subsequently contacting the substrate with at least one polar solvent to elute the olefinic hydrocarbons from the substrate in an olefinic fraction. The at least one substrate can include a silver ion loaded strong cation exchange column.

Separating can further include separating substantially all saturated hydrocarbons from the sample into a saturates fraction. To separate saturated hydrocarbons, the method can include contacting the hydrocarbon sample with a substrate having preferential affinity for unsaturated, nonolefinic hydrocarbons (such as aromatic hydrocarbons with two or more ring structures, polar hydrocarbons, and sulfide species) to immobilize the unsaturated, nonolefinic hydrocarbons on the substrate exhibiting preferential affinity for the unsaturated, nonolefinic hydrocarbons. Contacting the hydrocarbon sample with the substrate exhibiting preferential affinity for unsaturated, nonolefinic hydrocarbons can occur before contacting the hydrocarbon sample with the substrate exhibiting preferential affinity for olefinic hydrocarbons. The methods can further comprise preparing the hydrocarbon sample for preparation, such as by dissolving the hydrocarbon sample in a nonpolar organic solvent.

Spectroscopically measuring proton resonance signals of the olefinic fraction can comprise detecting chemical shift signal data of the hydrocarbons in the olefinic fraction in a proton NMR spectrometer. Quantifying the olefinic hydrocarbons based at least in part on the proton resonance signals of the olefinic fraction can include correlating the chemical shift signal data of the olefinic fraction with one or more known chemical shifts associated with olefinic hydrocarbons, and can additionally include integrating the chemical shift signal data of the hydrocarbons in the olefinic fraction to generate integrated olefinic hydrocarbon chemical shift signal data for each of the one or more chemical shifts associated with olefinic hydrocarbons. The one or more known chemical shifts associated with olefinic hydrocarbons can be a plurality of known chemical shifts, wherein two or more of the known chemical shifts are associated with a different subtype of olefinic hydrocarbon having a different predicted number of alkyl substitutions. Quantifying the olefinic hydrocarbons can be based at least in part on the integrated olefinic hydrocarbon chemical shift signal data for each of the two or more subtypes of olefinic hydrocarbon and the predicted number of alkyl substitutions for each subtype of olefinic hydrocarbon.

Accordingly, in another aspect of the present disclosure, a non-transitory computer readable medium is provided comprising a set of executable instructions to direct a processor to obtain, from a proton nuclear magnetic resonance spectrometer, data representing a proton chemical shift spectrum for a fraction of olefinic hydrocarbons separated from a hydrocarbon sample, to identify, from the data representing the proton chemical shift spectrum, based on known chemical shifts for olefinic hydrocarbons, chemical shift signal data corresponding one or more chemical shifts characteristic of olefinic hydrocarbons, to integrate the chemical shift data for each of the one or more chemical shifts characteristic of an olefinic hydrocarbon, and to quantify the olefinic hydrocarbons in the hydrocarbon sample based at least in part on the integrated chemical shift signal data for each the one or more chemical shifts characteristic of an olefinic hydrocarbon.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the disclosed subject matter. Together with the description, the drawings serve to explain the principles of the disclosed subject matter.

### BRIEF DESCRIPTION OF THE DRAWINGS

FIG. 1A is a flow chart illustrating a representative method implemented according to an illustrative embodiment of the disclosed subject matter.

FIG. 1B is a flow chart illustrating a separating process of a representative method implemented according to an illustrative embodiment of the disclosed subject matter

FIG. 1C is a flow chart illustrating a measuring process of proton resonance signals by proton NMR spectroscopy of a representative method implemented according to an illustrative embodiment of the disclosed subject matter.

FIG. 1D is a flow chart illustrating a quantifying process of a representative method implemented according to an illustrative embodiment of the disclosed subject matter.

FIG. 2 is a block diagram and flow path of a representative separation system according to an illustrative embodiment of the disclosed subject matter.

FIG. 3 is a representative chromatographic separation trace demonstrating separation of olefinic hydrocarbons, saturated hydrocarbons, and unsaturated, nonolefinic hydrocarbons according to an illustrative embodiment of the disclosed subject matter.

FIG. 4 is a representative bar graph of the weight percentage of olefins in two hydrocarbon samples as determined by using the average number of carbon atoms per hydrocarbon molecule (C_{avg}) and the integrated peaks of the olefinic chemical shift signals in the proton NMR spectrum, the weight percent of olefins can be calculated using the following formula:

$$weight percentage of olefins = \sum ( ( \int olefins \times ( \frac{1 mol olefins}{number of mols olefinic protons} ) \div \int total protons \times ( \frac{1 mol sample}{2 mols protons \times Cavg} ) ) * 100 \% ) \qquad Formula I$$

FIG. 5A is a proton NMR spectrum of a 50 mg hydrocarbon sample that was reconstituted after separation according to an illustrative embodiment of the disclosed subject matter.

FIG. 5B is a proton NMR spectrum of an isolated fraction of saturated hydrocarbons from the 50 mg hydrocarbon sample of FIG. 5A.

FIG. 5C is a proton NMR spectrum of an isolated fraction of olefinic hydrocarbons from the 50 mg hydrocarbon sample of FIG. 5A.

FIG. 5D is a proton NMR spectrum of an isolated fraction of unsaturated, nonolefinic hydrocarbons from the 50 mg hydrocarbon sample of FIG. 5A.

FIG. 6 is a chromatographic separation trace of a 160 mg hydrocarbon sample demonstrating separation of olefinic hydrocarbons from saturated hydrocarbons, and unsaturated, nonolefinic hydrocarbons according to an illustrative embodiment of the disclosed subject matter.

### DETAILED DESCRIPTION OF THE PREFERRED EMBODIMENT

Reference will now be made in detail to the various exemplary embodiments of the disclosed subject matter, exemplary embodiments of which are illustrated in the accompanying drawings. The structure and corresponding system of the disclosed subject matter will be described in conjunction with the detailed description of the method.

The methods and systems presented herein can be used for quantification of the olefins in a hydrocarbon sample. The disclosed subject matter is particularly suited for sensitive quantification of the weight percentage of olefins in a hydrocarbon sample as well as the relative abundance of various subtypes of olefinic hydrocarbons in the sample.

As used herein, the term “a hydrocarbon sample” will refer to one or more samples of a hydrocarbon stock, including, without limitation, a single sample of a hydrocarbon stock, multiple samples of the same hydrocarbon stock, a sample of a single hydrocarbon stock that is subdivided into a plurality of subsamples, as well as a plurality of samples of a plurality of hydrocarbon stocks. The term is also used to refer to a residual or remaining hydrocarbon sample after one or more portions of the sample are separated from the hydrocarbon sample, such as by contacting the hydrocarbon sample with a substrate.

As used herein, the terms “olefin,” “olefinic,” “olefins,” and “olefinic hydrocarbons” will refer generally and interchangeably to aliphatic hydrocarbons containing at least one double bond between adjacent carbon atoms. The term “saturated hydrocarbons” will generally refer to aliphatic hydrocarbons containing no double bonds between adjacent hydrocarbons. The term “unsaturated, nonolefinic hydrocarbons” will generally refer to hydrocarbons that are neither olefinic as referred to herein nor saturated as referred to herein. For example, the term “unsaturated, nonolefinic hydrocarbons” can refer to single or multi-ring aromatic hydrocarbons. As used herein, the terms “subtype of hydrocarbons” will generally refer to one of olefinic hydrocarbons, saturated hydrocarbons, or unsaturated, nonolefinic hydrocarbons.

Due, however, to the chemical similarity and overlap between signals (chemical shifts) for single-ring aromatic hydrocarbons and aliphatic olefins, where reference is made to fractions of olefins and/or olefinic hydrocarbons and/or to proton NMR spectra and/or data derived therefrom, such fractions, spectra and data may also include trace single-ring aromatic hydrocarbons and associated proton NMR signals.

Separating a subtype of hydrocarbons, such as olefinic hydrocarbons, from a hydrocarbon sample can comprise separating substantially all of the subtype from the hydrocarbon, or can comprise separating a known or predicted proportion of the subtype of hydrocarbons from the hydrocarbon sample. As embodied herein, separating olefinic hydrocarbons from a sample can comprise separating substantially all olefinic hydrocarbons from the hydrocarbon sample.

In accordance with the disclosed subject matter herein, a method for quantification of olefinic hydrocarbons can include providing a hydrocarbon sample, separating olefinic hydrocarbons from the sample into an olefinic fraction, spectroscopically measuring the proton resonance signals of the olefinic fraction, and quantifying the olefinic hydrocarbons based at least in part on the proton resonance signals of the olefinic fraction.

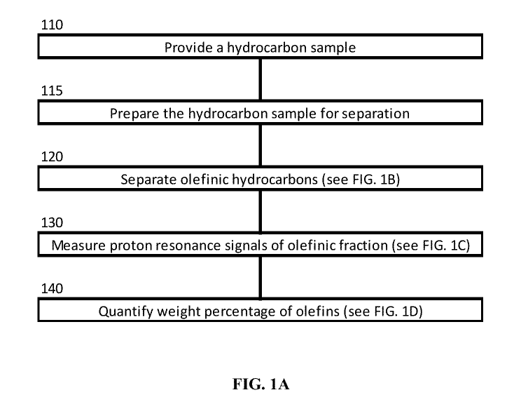

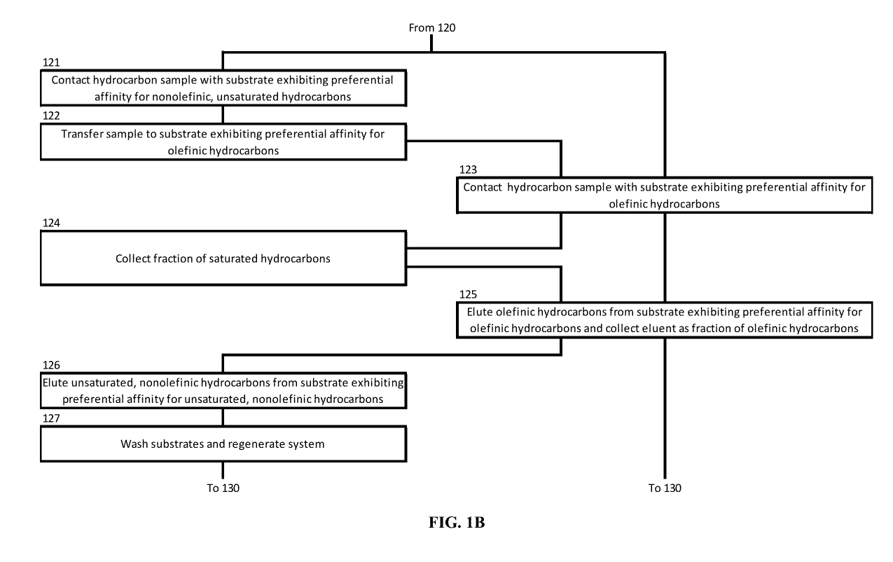

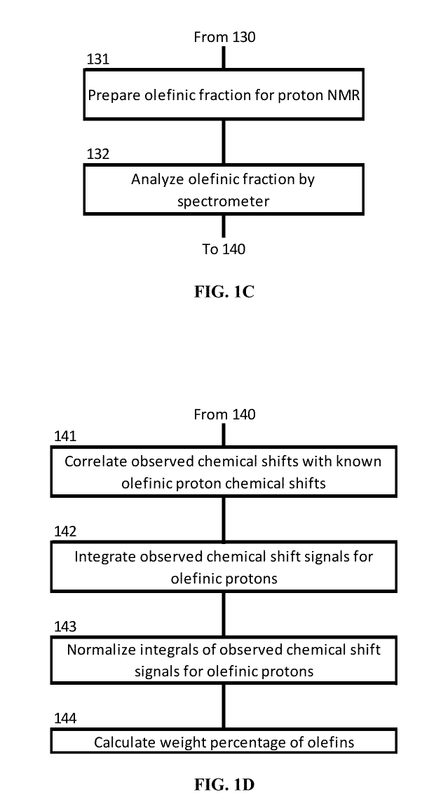

The accompanying figures, where like reference numerals refer to identical or functionally similar elements throughout the separate views, further illustrate various embodiments and explain various principles and advantages all in accordance with the disclosed subject matter. For purpose of explanation and illustration, and not limitation, exemplary embodiments of methods and systems for quantifying olefins in a hydrocarbon sample in accordance with the disclosed subject matter are shown in FIGS. 1-6. While the present disclosed subject matter is described with respect to quantification of olefins in a hydrocarbon sample, one skilled in the art will recognize that the disclosed subject matter is not limited to the illustrative embodiment. For example, the disclosed methods and systems can be used for a wide variety of hydrocarbon compositional quantification applications, such as quantification of the saturated hydrocarbons in a hydrocarbon sample and/or quantification of unsaturated, nonolefinic hydrocarbons in a sample, including aromatic hydrocarbons with one or more ring structures, polar hydrocarbons, and sulfide species.

FIG. 1A is a flow chart illustrating a representative method implemented according to an illustrative embodiment of the disclosed subject matter. Referring to FIG. 1A, at 110, a hydrocarbon sample is provided. The hydrocarbon sample can be a sample of any kind of hydrocarbons. As embodied herein, the hydrocarbon sample can be a sample of a hydrocarbon stock of high molecular weight hydrocarbons. By way of example, and not limitation, the hydrocarbon sample can be a vacuum gas oil or “heavy” petroleum stream, with a boiling point between about 550° F. and about 1050° F. The hydrocarbon sample can be, for example, an aliquot of a feedstock under evaluation for its suitability as a finished hydrocarbon product, such as a lubricant. Suitable feedstocks include, without limitation, virgin crude hydrocarbons, synthetic crude hydrocarbons, and processed feedstock hydrocarbons.

The amount of the hydrocarbon sample can be selected to permit accurate quantification of olefins in the sample. As embodied herein, the amount of hydrocarbon sample can have a mass of about 50 mg or greater, or a mass of about 100 mg or greater, or about 160 mg or greater, or about 320 mg or greater. As discussed in the examples below, a hydrocarbon sample can be divided and the olefinic concentration of each subsample can be combined or assayed in replicate (e.g., in duplicate). Olefinic fractions from each subsample can then be combined to increase detection of olefinic signals by proton NMR, such as by improving the signal-to-noise ratio of proton NMR detection.

At 115, the hydrocarbon sample is prepared for separation. In the exemplary embodiments disclosed, separation is performed by high-pressure liquid chromatography (“HPLC”), as further described below. It is noted, however, that any suitable alternative method of separating olefinic hydrocarbons from the hydrocarbon sample is contemplated by the present disclosure.

Accordingly, and as embodied herein, separation is performed using a HPLC apparatus. The HPLC apparatus can be configured to separate a series of samples in sequence, or to separate aliquots (i.e., subsamples) of a single hydrocarbon sample in sequence. Thus, multiple samples can be prepared for separation by an operator at the beginning of a sampling operation or workflow as desired.

As embodied herein, preparing the sample for separation can include dissolving the hydrocarbon sample in an appropriate volume of a suitable organic solvent. The sample can be warmed and agitated to ensure complete dissolution in a suitable solvent. Suitable solvents include non-polar organic solvents, such as hexane, [heptane, toluene, cyclohexane and combinations (e.g., mixtures) thereof. The sample or samples can be prepared for separation, such as by dissolution in solvent. At the completion of 115, the hydrocarbon sample thus can be prepared for separation of olefinic hydrocarbons. In the embodiments disclosed herein, the prepared hydrocarbon sample is a hydrocarbon sample that is dissolved in an appropriate volume of a suitable solvent, as described.

With further reference to FIG. 1A, at 120, olefinic hydrocarbons are separated from the prepared hydrocarbon sample. In an exemplary embodiment, separation of olefinic hydrocarbons from the prepared hydrocarbon sample proceeds by contacting the prepared hydrocarbon sample with a substrate having preferential affinity for olefinic hydrocarbons in the prepared hydrocarbon sample. As used herein, the term “a substrate with a preferential affinity for olefinic hydrocarbons in the prepared hydrocarbon sample” refers to material that selectively binds olefinic hydrocarbons in the prepared hydrocarbon sample. As embodied herein, a single substrate can selectively (e.g., exclusively) bind the olefinic hydrocarbons in the prepared hydrocarbon sample for elution as an isolated fraction of the hydrocarbon sample.

In alternative embodiments, one or more, or two or more substrates can be used to sequentially bind and remove components of the hydrocarbon sample, such as hydrocarbon subtypes having similar chemical affinity to the substrate in a selected solvent or solvent mixture. For example, a first column can contain a substrate having preferential affinity for unsaturated, nonolefinic hydrocarbons in the hydrocarbon sample, and a second column can contain a substrate having preferential affinity for olefinic hydrocarbons in the hydrocarbon sample. Unsaturated hydrocarbons can be eluted in a fraction by first transferring unbound hydrocarbons from the hydrocarbon sample (i.e., unsaturated and olefinic hydrocarbons) from the first column to the second column, then collecting the unsaturated hydrocarbons from the second column. The olefinic hydrocarbons can then be eluted from the second column with a solvent or solvent mixture. The unsaturated, nonolefinic hydrocarbons also can be eluted from the first column by rinsing the first column with a solvent or solvent mixture.

As embodied herein, separation of olefinic hydrocarbons from a hydrocarbon sample can proceed by running the selected, prepared hydrocarbon sample through a HPLC apparatus having one or more chromatography columns containing a substrate having preferential affinity for a subset of hydrocarbons to be separated (whether actually present or not in the prepared sample) such as olefinic hydrocarbons. In embodiments where a single column is employed, the chromatography column can contain a substrate with preferential affinity for olefinic hydrocarbons. Where two or more chromatography columns are employed, one or more of the columns can contain a substrate with preferential affinity for olefinic hydrocarbons.

As embodied herein, the one or more columns can contain a substrate that exhibits affinity for olefinic hydrocarbons as well as certain nonolefinic hydrocarbons in the presence of a selected solvent or solvent mixture, and exhibits a preferential affinity for nonolefinic hydrocarbons in the presence of another selected solvent or solvent mixture. The olefinic hydrocarbons can be selectively eluted from the substrate by contacting the hydrocarbons bound on the substrate with a selected solvent or solvent mixture to remove only or substantially only olefinic hydrocarbons from the substrate. Alternatively, the one or more columns can contain a substrate that exhibits affinity for olefinic hydrocarbons as well as certain nonolefinic hydrocarbons in the presence of a selected solvent or solvent mixture, and exhibits a preferential affinity for olefinic hydrocarbons in the presence of another selected solvent or solvent mixture. The nonolefinic hydrocarbons can be selectively eluted from the column by contacting the hydrocarbons bound on the substrate with a selected solvent or solvent mixture, leaving the purified olefinic hydrocarbons bound on the substrate for subsequent elution.

As embodied herein, it can be advantageous to employ two or more columns, two or more of the columns containing a substrate exhibiting preferential affinity for a separate chemical subtype of hydrocarbons that may be present in the hydrocarbon sample. By way of example and not limitation, one column can contain a substrate that exhibits preferential affinity for unsaturated, nonolefinic hydrocarbons (such as multi-ring aromatic hydrocarbons), while a second column can contain a substrate that exhibits preferential affinity for olefinic hydrocarbons. By contacting the hydrocarbon sample with the first and second substrates, the hydrocarbon sample can be separated into three fractions, one containing saturated hydrocarbons, one containing olefinic hydrocarbons, and one containing unsaturated, nonolefinic hydrocarbons.

While explicit reference is made herein to first and second columns, it will be readily understood that additional and/or alternative embodiments can employ third, fourth, and additional columns having the same or functionally similar substrates as described for the first and second columns.

In practice, it has been found that available chromatography substrates can exhibit affinity to both olefinic hydrocarbons and certain unsaturated nonolefinic hydrocarbons. As embodied herein, the olefinic hydrocarbons along with some 1-ring aromatics can be selectively eluted from a chromatographic substrate by the use of one or more solvents. Additionally or alternatively, olefinic hydrocarbons can be purified of unsaturated nonolefinic hydrocarbons by selective elution of the olefinic hydrocarbons with one or more solvents. Polar solvents in particular can selectively remove bound hydrocarbons from the substrate, and the polarity of the solvent or solvent mixture can be selected to selectively remove bound hydrocarbons based on known or expected strength of binding to the substrate. As embodied herein, the column can be rinsed with a solvent gradient selected to increase or decrease in polarity over the duration of an elution to selectively remove bound hydrocarbons of a given subtype from the substrate. The olefinic hydrocarbons are concentrated in the olefins fraction and the olefins fraction is contaminated with some saturates and 1-ring aromatics hydrocarbons.

Where reference is made herein to a solvent, it will be understood that “a solvent” can include a single solvent as well as a combination, such as a mixture, of two or more solvents. Similarly, where reference is made herein to rinsing with a solvent, it will be understood that rinsing will include a single rinse with a single solvent, a single rinse with a combination of two or more solvents, two or more rinses with a single solvent, two or more rinses with two or more separate solvents, two or more rinses with two or more combinations of two or more solvents, and the like.

Thus, in exemplary embodiments, and with reference to FIG. 1B, the separation 120 can proceed at 121 by contacting the prepared hydrocarbon sample with a substrate in a first chromatography column with affinity for a first subtype of hydrocarbons, which can be unsaturated nonolefinic hydrocarbons. At 122, the olefinic hydrocarbons and saturated hydrocarbons of the hydrocarbon sample are transferred to a second chromatography column. At 123, the olefinic hydrocarbons and saturated hydrocarbons of the hydrocarbon sample contact a substrate in the second chromatography column. The substrate in the second chromatography column can exhibit preferential affinity for olefinic hydrocarbons. At 124, the unbound saturated hydrocarbons are rinsed from the second column and collected. At 125, the olefinic hydrocarbons are eluted from the substrate exhibiting preferential affinity for the olefinic hydrocarbons by backflushing the second column with a solvent, such as a polar solvent mixture, and collected. If any residual nonolefinic hydrocarbons left in the second column and the olefinic hydrocarbons can be selectively eluted by rinsing with a suitable solvent, such as a polar solvent mixture. At 126, the first column is backflushed to elute the unsaturated nonolefinic hydrocarbons from the substrate for detection, collection and/or disposal. At 127, the substrates in the columns are washed and regenerated.

With further reference to FIG. 1B, an alternative separation process is shown. At 123, the hydrocarbon sample is contacted with a substrate exhibiting preferential affinity for olefinic hydrocarbons, and at 125, the olefinic hydrocarbons are eluted from the substrate exhibiting preferential affinity for olefinic hydrocarbons, and the eluent is collected as a fraction of olefinic hydrocarbons. The olefinic hydrocarbons can be eluted by contacting the substrate with a suitable solvent, such as a polar solvent mixture.

It will be understood that the specific sequences disclosed above are in no way limiting, and that the chromatographic separation of hydrocarbon samples can proceed in a different sequence and/or with additional intervening processes. While the various processes of the method are disclosed as separate operations, certain processes of the exemplary method described can proceed substantially continuously from one process to the next. Moreover, certain processes of the exemplary sequence described can be performed simultaneously or contemporaneously.

Furthermore, as embodied herein, the HPLC apparatus can be configured to automatically or semi-automatically separate olefinic hydrocarbons from the hydrocarbon sample into an olefinic fraction. The HPLC apparatus can further be configured to automatically or semi-automatically separate one or more additional fractions from the hydrocarbon sample, such as a saturated hydrocarbons fraction and a fraction containing unsaturated and non-olefinic hydrocarbons, such as aromatic hydrocarbons with two or more ring structures, polar hydrocarbons, and sulfide species.

FIG. 2 is a schematic diagram of an exemplary separation system, according to an illustrative embodiment of the disclosed subject matter. Particularly, the exemplary system of FIG. 2, for purpose of illustration and not limitation, is a HPLC apparatus and discussed further with reference to the exemplary separation method of FIG. 1B. In FIG. 2, an exemplary HPLC apparatus having a first valve 211 in fluid communication with a second valve 212 is depicted. One or more valves 211, 212 can be in fluid communication with a solvent delivery unit 215. Two chromatography columns 221, 222 (the dimensions of each of the columns are 250 mm×10 mm, but other column dimensions are considered to be well within the scope of the present invention) in fluid communication with the first valve 211 and second valve 212 are shown. The second valve 212 is in fluid communication with a third valve 213 which is set up in the thermostat chamber of the HPLC system. Any valve will be acceptable long as the valve can be switched between 2 positions with at least 4 ports (shown with 6 ports). The valve 213 is in fluid communication with an evaporative light scanning detector (ELSD) 230 and a fraction collector module 240. The apparatus contains one or more flow paths 250 between the valves. As depicted, an optional ultraviolet detector 260 is also provided in the flow path between the second valve 212 and third valve 213.

At 120, a prepared hydrocarbon sample is introduced into a first valve 211, such as by injection, into a solvent stream in a flow path 250. The solvent can be the same as the solvent used to dissolve the hydrocarbon sample during sample preparation. The solvent stream can have any suitable flow rate. For example, the solvent flow rate can be about 10 mL/min. At 121, the hydrocarbon sample is directed in the solvent stream along a flow path 250 to a first chromatography column 221. First chromatography column 221 can contain a first chromatography substrate exhibiting preferential affinity for a first subtype of hydrocarbons that may be present in the hydrocarbon sample. In the disclosed exemplary embodiments, the first subtype of hydrocarbons can be unsaturated nonolefinic hydrocarbons, such as multi-ring aromatic hydrocarbons, and the chromatography substrate can be 2,4-dinitro-anilino-propyl silica gel (DNAP) which is very selective for multi-ring aromatics. Alternatively, the generic substrates such as silica gel and alumina having some affinity for unsaturated nonolefinic hydrocarbons can also be used.

The hydrocarbon sample can be directed continuously or substantially continuously through the first column, where the hydrocarbon sample contacts the first chromatography substrate. At 122, the hydrocarbons remaining in the hydrocarbon sample after contacting the first chromatography substrate (e.g., olefinic hydrocarbons and saturated hydrocarbons) are eluted from the first column. At 123, the hydrocarbons remaining in the hydrocarbon sample after contacting the first chromatography substrate are directed via flow path 250 and second valve 212 to the second chromatography column 222 to contact a second chromatography substrate having preferential affinity for a second subtype of hydrocarbons that may be present in the hydrocarbon sample. In the disclosed exemplary embodiments, the second subtype of hydrocarbons can be olefinic hydrocarbons, and the chromatography substrate can be a silver ion-loaded strong cation exchange resin. Alternatively, other substrates such as silver-ion, palladium-ion or some other heavy metal-ion loaded silica gel and/or alumina having some affinity for olefins can also be used.

At 124, a first fraction is washed from second column 222 by continued flow of the solvent stream through the second column. The first fraction can be directed to third valve 213 via flow path 250, can be detected by a detector, such as ultraviolet (UV) light detector 260 and ELS detector 230 or can be detected by UV detector and then collected by a fraction collector 240 depending upon the valve 213 switching position. In the disclosed exemplary embodiments, the first fraction consists substantially entirely of saturated hydrocarbons. Steps 122 and 123, 123 and 124, and 122 and 124 can occur substantially continuously and or contemporaneously, and without interruption of the solvent stream. In the disclosed exemplary embodiments, 122, 123, and 124 can occur in a timespan of about 5.5 minutes. Such timespan will be influenced by a number of variables, including sample volume, column size, and flow rate.

At 125, the direction of flow of solvent is reversed and the second column 212 and chromatographic substrate are rinsed (i.e., backflushed) with a solvent mixture, such as a polar solvent mixture. In the exemplary embodiments disclosed, the solvent mixture can be a 75:5:20 percent by weight mixture of methylene chloride, methanol, and toluene. Subsequently, the second fraction can be eluted by a brief rinse with an additional solvent mixture, such as and the final solvent mixture can be a 90:10 percent by weight mixture of methylene chloride and methanol. The eluent from the second column can be detected by UV detector 260 and ELS detector 230 or can be detected by UV detector and then collected by a fraction collector 240 as a second fraction. In the disclosed exemplary embodiments, the second fraction consists substantially entirely of olefinic hydrocarbons. Trace unsaturated nonolefinic hydrocarbons may also be present in the second fraction.

At 126, the first column is also back flushed with a solvent mixture, such as a polar solvent mixture. The solvent mixture can be a solvent mixture gradient with an initial composition of 90:10 percent by volume methylene chloride and methanol, and a final solvent composition of 70:10:20 percent by volume methylene chloride, methanol, and toluene. The first column eluent can be detected by UV detector 260 and ELS detector 230 or can be detected by UV detector and then collected by a fraction collector 240 as a third fraction of unsaturated nonolefinic. In the exemplary embodiments, the timespan for back flushing of the first column is about 3.0 minutes.

At 127, first and second columns 221, 222 and first and second substrates are cleaned by rinsing with polar solvent, such as methylene chloride. In the disclosed exemplary embodiments, the second column 222 is rinsed for two minutes and the first column 221 is rinsed for ten minutes. Rinsing of the second column 222 can occur prior to back flushing and rinsing of the first column 221 to elute the third fraction. After the columns are rinsed, the columns and substrate can be permitted to regenerate for at least 20 minutes with nonpolar solvent to equilibrate the columns before commencing a subsequent separation. This regeneration process brings the HPLC system including the HPLC columns performance efficiency to its initial separation step.

With further reference to the exemplary method of FIG. 1A, at 130, the proton resonance signals of the olefinic fraction of the hydrocarbon sample are spectroscopically measured. The resonance signals from the olefinic fraction of a single separation run can be spectroscopically measured, or the olefinic fractions collected during separation of a plurality of hydrocarbon samples or subsamples can be combined for spectroscopic measurement. Depending on sample volume, it can be advantageous to combine the olefinic fractions collected during separation of a plurality of hydrocarbon samples or subsamples. In the disclosed exemplary embodiments, a combined total sample mass of about 100 mg or greater, or about 300 mg or greater, over two separation runs (i.e., by combining the olefinic fractions collected from two sample separations) was found to permit accurate quantification of olefins with a weight percentage in the hydrocarbon sample as low as 0.2. For hydrocarbon samples with an olefin content of about 1 percent by weight, the precision of quantification was found to be +/−0.04 percent by weight for a hydrocarbon sample having a total or combined mass of about 300 mg.

With reference now to FIG. 1C, at 131, the olefinic fraction of a hydrocarbon sample is prepared for proton NMR spectroscopy. As embodied herein, the olefinic fraction is prepared for proton NMR spectroscopy by diluting the samples to a concentration of 5-10% by volume in deuterated chloroform (CDCl₃) with 0.03% tetramethylsilane as a zero ppm reference.

At 132, the prepared olefinic fraction is analyzed in a proton NMR spectrometer. Olefinic protons resonate in the chemical shift region between about 4.0 to about 6.5 ppm. Accordingly, the proton NMR spectrometer can be configured to detect chemical shifts under a homogenous magnetic field in the region of between about 4.0 ppm to about 6.5 ppm.

The resulting proton NMR spectrum can display one or more resonance (i.e., chemical shift) signal peaks each corresponding to a signal of an olefinic proton. There are five distinctive proton NMR chemical shifts each associated with a different type of olefinic proton as shown in Table 1 below. Each type of olefinic proton is characteristic of a different subtype of olefinic hydrocarbon (e.g., aliphatic, aromatic, different alkyl substitution pattern), and, as indicated by the number of R-substituents in Table 1, the various subtypes of olefinic hydrocarbons can have different numbers of alkyl substitutions. As used herein, the term “subtype of olefinic hydrocarbon” will refer to one of the five possible olefinic hydrocarbons distinguishable on the basis of its proton NMR chemical shift as shown in Table 1 below.

TABLE 1
Chemical Shifts for Olefins Based on
Double Bond Location and Substitution

| Double Bond Location | proton Chemical Shift (ppm) | Reference |
|---|---|---|
| RCH═CH₂ | 6.0-5.6 | A |
| RCH═CHR | 5.6-5.2 | B |
| RCH═CR₂ | 5.2-5.0 | C |
| RCH═CH₂ | 5.0-4.8 | D |
| R₂C═CH₂ | 4.8-4.6 | E |

In Table 1, reference A & D are the same molecule, but the different protons have different chemical shifts due to different chemical environment. The average carbon number can be estimated from the 50% off point of the Simulated Distillation of the hydrocarbon sample by correlating to the boiling point of the normal paraffin.

At 140, as shown in FIG. 1A, the weight percentage of olefins in the hydrocarbon sample is determined. The proton NMR spectrum data indicating the chemical shift signal data for the olefinic fraction can be used to calculate the weight percentage of olefinic hydrocarbons in the hydrocarbon sample. Accordingly, and with reference now to FIG. 1D, methods of quantifying olefinic hydrocarbons in a hydrocarbon sample based at least in part on proton NMR chemical shift signal data from the proton NMR spectrum of an olefinic fraction of the hydrocarbon sample are provided. At 141, the chemical shift signal data from the proton NMR spectrum are correlated with the chemical shifts for olefinic protons in olefinic hydrocarbons. At 142, the chemical shift signals for the olefinic protons are integrated. At 143, the integrals of the observed olefinic proton chemical shifts are normalized to 100. At 144, the weight percentage of olefins is calculated.

Using the average number of carbon atoms per hydrocarbon molecule (C_{avg} and the integrated peaks of the olefinic chemical shift signals in the proton NMR spectrum, the weight percent of olefins can be calculated using the following formula:

$$weight percentage of olefins = \sum ( ( \int olefins \times ( \frac{1 mol olefins}{number of mols olefinic protons} ) \div \int total protons \times ( \frac{1 mol sample}{2 mols protons \times Cavg} ) ) * 100 \% ) \qquad Formula I$$

The “∫ olefins” is the integrated peaks of the olefin signals from the NMR spectrum, the equivalent of the area under the peaks. Different olefinic hydrocarbons would have different number of olefinic proton in each molecule, and that number is the number of moles of olefinic protons. For example, in Table 1, B has 2 moles of olefinic protons per olefins molecule and C has 1 mole of olefinic proton per olefin molecule. Assuming that the average olefinic molecule in the sample is an aliphatic olefins with no naphthenic ring, the total number of protons in the molecule is 2* the average carbon number.

As indicated in Table 1, each type of olefinic proton is associated with a different subtype of olefinic hydrocarbon. The various subtypes can have different numbers of alkyl substitutions. For example, olefinic protons having a chemical shift at the 5.0-5.2 ppm range are associated with a subtype of olefinic hydrocarbons having three alkyl substitutions, whereas olefinic protons having a chemical shift at the 6.0-5.6 ppm range are associated with a subtype of olefinic hydrocarbons having one alkyl substitution. The number of alkyl substitutions in an olefinic hydrocarbon will correlate directly with its molecular mass. Accordingly, in some embodiments of the present disclosure, quantifying the olefinic hydrocarbons in a hydrocarbon sample can include identifying chemical shift signal data corresponding to two or more subtypes of olefinic hydrocarbons having different numbers of alkyl substitutions, and the quantifying can be based at least in part on the quantity of each subtype of olefinic hydrocarbons.

Thus, as embodied herein, the integrals of the chemical shift signal data for each type of olefinic proton (as set forth in Table 1 above) can be included to account for the different number of olefinic protons in the various subtypes of olefinic hydrocarbons using the following formula:

weight percent of olefins=(∫ Peak C)×2×(Cavg)+(∫ Peak B+∫ Peak E)×2×(Cavg)+(∫ Peak A+∫ Peak D)×1.5×(Cavg) Formula II

Additionally or alternatively, the weight percentage of olefins in the hydrocarbon sample can be calculated from the proton NMR spectrum observed for the olefinic hydrocarbons separated from the hydrocarbon sample. Where the ∫Peaks are the integrated peak area values of the respective peaks in the NMR spectrum. The integrated values of the total spectrum are normalized to 100.

Furthermore, the proton NMR spectrum can be used to determine the relative abundance of each type of olefin as referenced in Table 1 (e.g., aromatic, aliphatic, and olefinic). The integrated area of each of the olefinic peaks over the total proton signal in the samples provides the relative abundances of each of the olefinic subtype in mole % relative to the total number of proton integrated in the sample. The weight % of the each olefinic type can then be calculated using the formula in Formula I, described above, or Formula II, described above, while only calculating the olefins of interest. The different olefinic subtypes should have different reactivity towards oxidation, so knowing the abundance of the olefinic subtypes should provide some indication of the oxidative performance of the sample. The weight % of the third sample is mainly to provide mass balance of the HPLC separation. There is not too much information that can be extracted from the HPLC separation or the NMR of that fraction. Typically, further separation of that third fractions and even the olefinic fraction is necessary to provide fractions for subsequent analysis to determine the composition.

In accordance with another aspect of the disclosed subject matter, a system for quantifying olefins in a hydrocarbon sample can include one or more processors. For example, the processor can include non-transitory computer readable storage media embodying software to perform some or all of the method disclosed herein. The processor(s) can be configured when executed by one or more of the processors to operate an HPLC apparatus, such as by valve switching at specified timing intervals to thereby separate a hydrocarbon sample into one or more fractions. As embodied herein, the processor(s) can be configured to perform one or more processes including directing a hydrocarbon sample to one or more chromatography columns, directing a fraction of a hydrocarbon sample to a fraction collector, directing a fraction of a hydrocarbon sample to a detector, and directing a polar solvent or a mixture of polar solvents to a chromatography column to elute a fraction of a hydrocarbon sample from a substrate having preferential affinity for the fraction. The processor(s) can be configured when executed to implement a sequence of valve switching and flow direction to execute the steps as depicted at FIG. 1B and described above.

Likewise, and in accordance with another aspect of the present disclosure, the proton NMR spectrometer can be equipped with one or more processors and with non-transitory computer readable storage media embodying software configured to acquire proton NMR signals from an olefinic fraction of a hydrocarbon sample. The processor(s) thus can be configured to correlate the proton NMR signals from an olefinic fraction of a hydrocarbon sample to one or more proton resonance NMR signals characterizing olefinic hydrocarbons, to integrate the proton resonance signals of the olefinic fraction, and/or to calculate the weight percentage of olefins in a hydrocarbon sample based at least in part on the proton resonance signals acquired from the olefinic fraction.

Accordingly, and as embodied herein, the processor(s) can be configured to obtain, from a proton nuclear magnetic resonance spectrometer, data representing a proton chemical shift spectrum for a fraction of olefinic hydrocarbons separated from a hydrocarbon sample, identify, from the data representing the proton chemical shift spectrum, chemical shift signal data corresponding one or more chemical shifts characteristic of olefinic hydrocarbons, integrate the chemical shift signal data for each of the one or more chemical shifts characteristic of an olefinic hydrocarbon, and quantify the olefinic hydrocarbons in the hydrocarbon sample based at least in part on the integrated chemical shift signal data for each the one or more chemical shifts characteristic of an olefinic hydrocarbon. Additionally, the processor(s) can be configured to instruct a processor to identify, from the data representing the proton chemical shift spectrum, chemical shift signal data corresponding to two or more chemical shifts each characteristic of a subtype of olefinic hydrocarbon having a different predicted number of alkyl substitutions and to quantify the olefinic hydrocarbons based at least in part on the integrated chemical shift signal data for each of the two or more chemical shifts each characteristic of a subtype of olefinic hydrocarbon and the predicted number of alkyl substitutions for each subtype of olefinic hydrocarbon.

The disclosed subject matter is further described by means of the examples, presented below. The use of such examples is illustrative only and in no way limits the scope and meaning of the disclosed subject matter or of any exemplified term. Likewise, the disclosed subject matter is not limited to any particular preferred embodiments described herein. Indeed, many modifications and variations of the disclosed

### EXAMPLES

An exemplary chromatographic separation protocol for separation of olefinic hydrocarbons from a hydrocarbon sample was developed using terminal (i.e., alpha-), internal olefin model compounds and hydrocarbon samples. 1-Eicosene, 9-heneicosene, saturated hydrocarbons, single-ring and multi-ring aromatic fractions of a vacuum gas oil distillate, and single-aromatic olefin fractions of vacuum gas oil range samples were used to optimize the exemplary protocol.

### Separation Protocol Validation

The exemplary protocol was tested to observe the specificity of separation of olefinic, saturated, and unsaturated nonolefinic hydrocarbon fractions from a hydrocarbon sample.

Separation was performed with an Agilent HPLC system equipped with an Agilent 1100 Series quaternary solvent delivery pump with a degasser, a thermostated column compartment with a 6-port switching valve, and two additional 10-port switching valves. The solvent delivery unit is programmable to deliver, for a specified duration at a specified flow rate up to 10 mL/min, a single solvent or two or more solvents in a mixture with a specified ratio. An Agilent 1200 Series fraction collector equipped with a funnel tray was used to collect the separated fractions from multiple runs. Ultraviolet (UV) and evaporative light scattering (ELS) detectors were used to record chromatographic separation traces. The HPLC apparatus included two 250 mm×10 mm HPLC columns, one loaded with 2,4-dinitro-anilino-propyl-silica gel and the other loaded with silver ion-loaded strong cation exchange resin. FIG. 2 is a typical block diagram of the HPLC system and its operational logic when the system is in detection mode with UV and ELS detection equipped. The valve switching method for the exemplary separation protocol is shown/provided in Table 2. The system was configured, through the thermostatic valve, to either pass the eluting fractions through a detectors for separation tracing for separation validation or to the fraction collector for collection in a first fraction (saturated hydrocarbons), a second fraction (olefinic hydrocarbons) and a third fraction (unsaturated, nonolefinic hydrocarbons) for further analysis. The flow rate was set to 10 mL/min.

TABLE 2
Switching Valves Positing during exemplary separation protocol.

| Time | Valve 1 | Valve 2 |
|---|---|---|
| 0.01 | 1 | 1 |
| 3.50 | 2 | 1 |
| 5.50 | 2 | 2 |
| 12.00 | 1 | 2 |
| 20.00 | 1 | 1 |

A hydrocarbon sample was prepared by dissolving a stock hydrocarbon aliquot to a concentration of about 130 mg/mL in hexane. Approximately 100 mg of sample in total was separated by injecting two 400 uL aliquots of the dissolved sample into the HPLC apparatus. The sample was directed through the DNAP column and then the silver ion loaded strong cation exchange column over a total timespan of 5.5 minutes in 100% hexane. In this manner, a first fraction was eluted from the Ag⁺SCX⁻ column between 2 and 6 minutes. The Ag⁺SCX⁻ column was then backflushed with a mixture of 75% methylene chloride, 5% methanol, and 20% toluene then changing to a mixture of 90% methylene chloride and 10% methanol over 4.5 minutes and followed with two minutes of 100% methylene chloride. The second fraction was eluted between 6 min and 11.5 min. The steps between 5.5 min and 6 min and 11.5 min and 12 min are the solvent gradients steps to elute the next fraction, Fraction 2. All the 0.01 second difference is for the timetable in the HPLC software to control gradient change. Subsequently, the DNAP column was back flushed starting with a mixture of 90% methylene chloride and 10% methanol, then changing to a mixture of 70% methylene chloride, 10% methanol, and 20% toluene over 3 minutes, and finally the column was washed with 100% methylene chloride for ten minutes. In this manner, the third fraction was eluted from the DNAP column between 11.5 and 18 minutes. Time 12.01 to 25.00 min includes solvent gradient steps and the third fraction is eluted during this time.

The total duration of each separation run was 25 minutes. The system was permitted to regenerate for 20 minutes between separation runs. Thus the total separation time per sample was 45 minutes. Accordingly, 16 or more samples can be separated unattended in a single day if the HPLC apparatus has a suitable fraction collector.

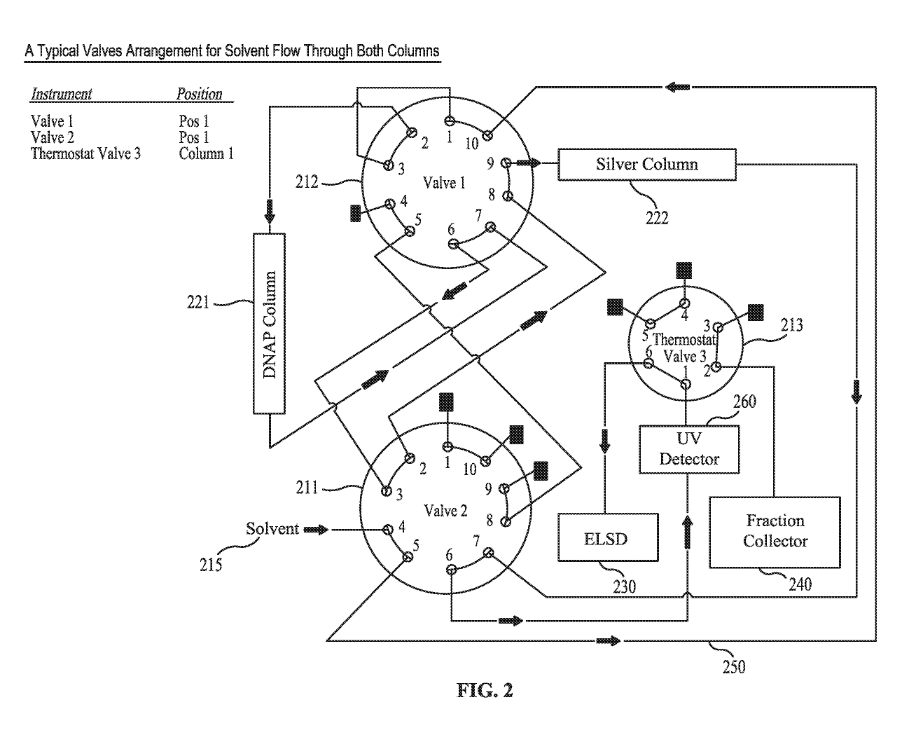

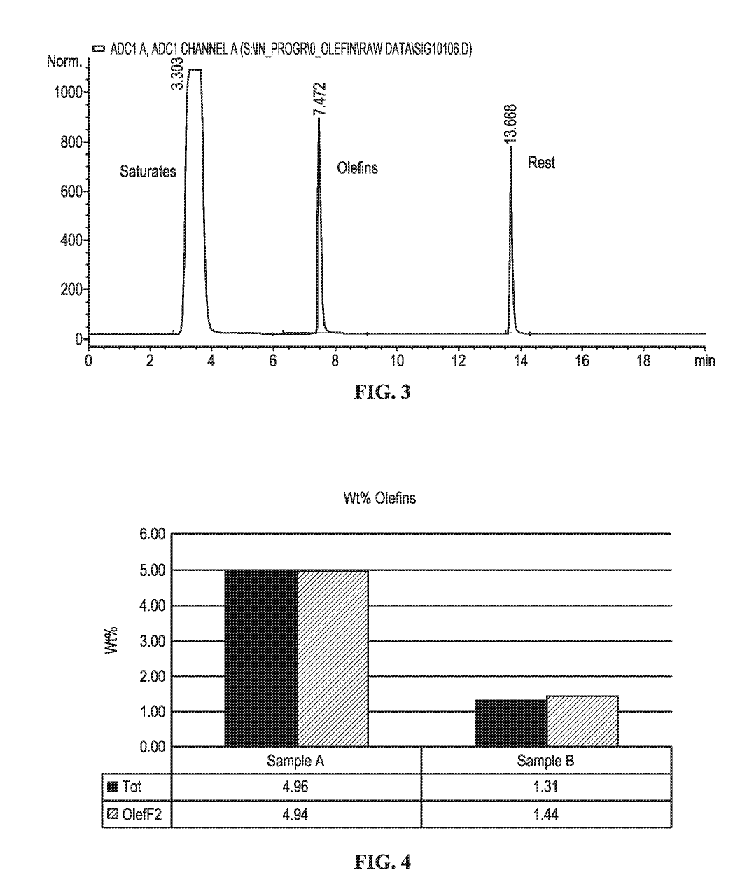

The chromatographic separation traces for the separation are provided in FIG. 3. The peaks are sharp and discrete, indicating excellent separation of each fraction. Each fraction from two separation runs was combined and the mass recoveries were determined by weighing the fractions after solvent evaporation. The total mass recovery was found to be about 98% of the mass of the injected sample. The weight percentages of each of the fractions are then normalized to 100% based on the % recovery of the fractions. Similar experiments during development of the exemplary separation protocol indicated that mass recovery was optimal for samples that did not contain any materials boiling below 550° F.

### Proton NMR Spectroscopy

Sample (e.g., fraction) solutions were prepared for proton NMR spectroscopy by diluting the samples to a 5- to 10% solution in deuterated chloroform (CDCl3) with 0.03% tetramethylsilane (TMS). The mass balanced fractions (after all solvent has been evaporated) are then diluted in deuterated chloroform and analyzed by running as standard proton NMR experiments. Proton NMR spectrometry was performed using for example a JEOL ECS400 spectrometer with the acquisition parameters as set forth in Table 3A and the data processing parameters as set forth in Table 3B. The chemical shifts are reported by reference to the TMS peak, which is set to zero.

TABLE 3A
Proton NMR Acquisition Parameters

| Parameter | Setting | |
|---|---|---|
| Spin state | SPIN ON | |
| Scans | 64 | |
| Relaxation Delay | 30 | seconds |
| x_offset | 5 | ppm |
| x_sweep | 15 | ppm |
| x_points | 32768 | |
| x_angle | 45 | degrees |

TABLE 3B
Proton NMR Data Processing Parameters

| Parameter | Setting |
|---|---|
| dc_balance | 0: False |
| Sexp | 0.2 Hz: 0.0 seconds |
| Transform | Fourier Transform |

### Separation Protocol Validation by NMR

The exemplary separation protocol was repeated with two base stock samples (Sample A and Sample B) and subsequently analyzing the separated fractions by proton NMR spectroscopy. The NMR spectra showed that the olefinic hydrocarbons fraction (i.e., the second fraction) contained only olefins, while no olefins were detected in the remaining fractions. The total amount of olefins present in the samples closely matched the olefins detected in the separated olefins fraction, as shown in FIG. 4. The total weight % olefin content of the sample is determined by the weight % olefins in the olefins fractions (as described above) and the normalized weight % recovery of the olefins fraction from the mass balance of the collected fractions from the HPLC elution.

### Sample Mass Optimization for NMR

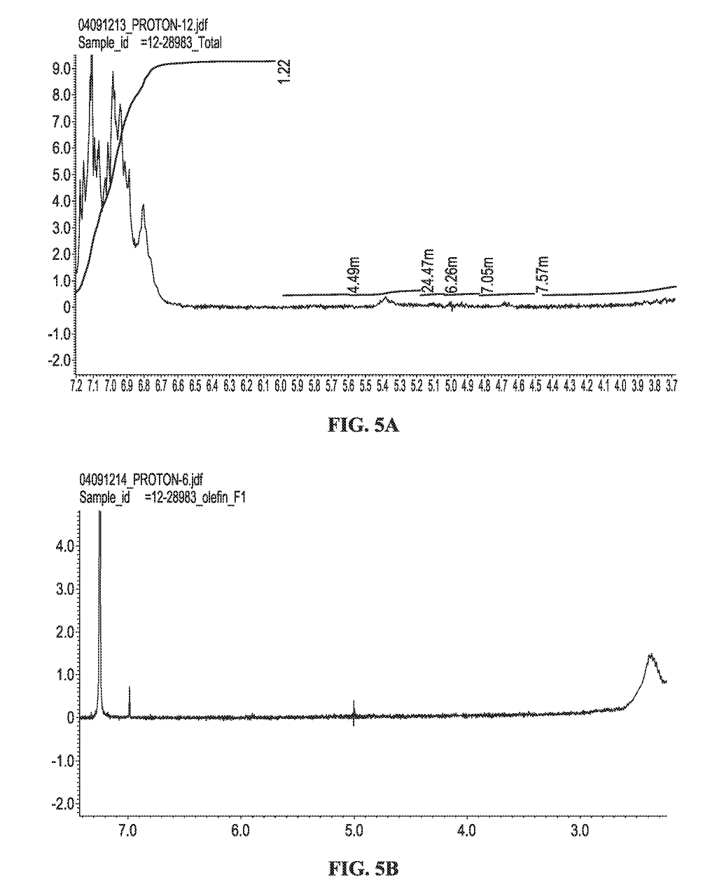

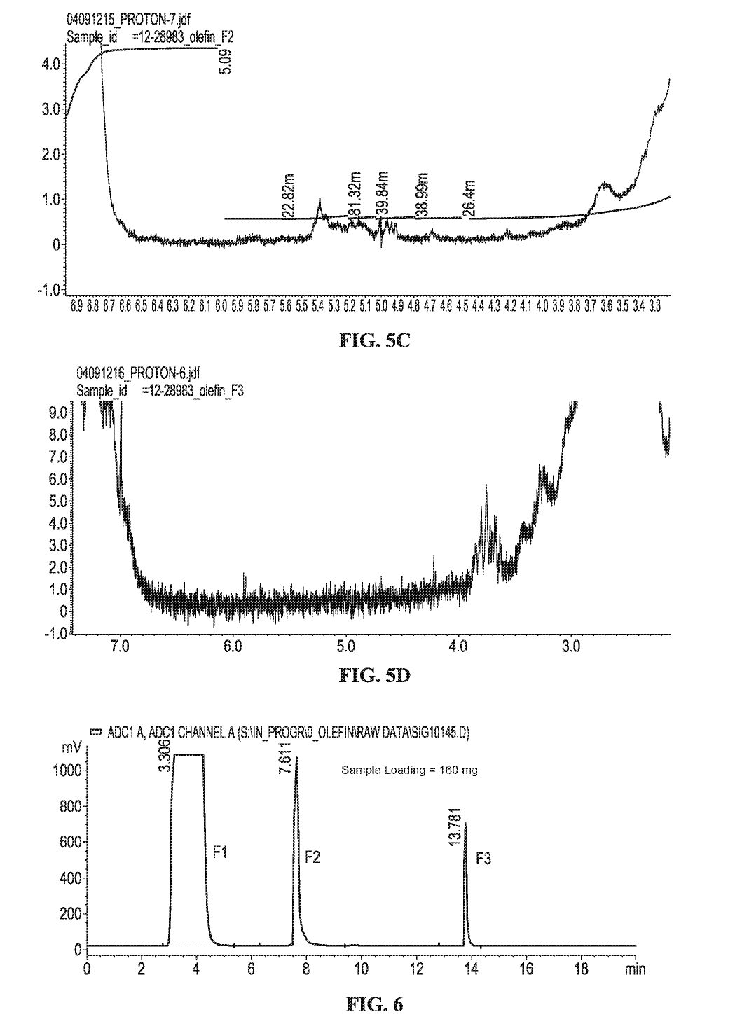

It was observed experimentally that the NMR signal-to-noise ratio can be too low to accurately measure the olefinic hydrocarbon content of samples containing less than about 1% olefins by weight using olefinic fraction collected by a single HPLC separation with 50 mg sample loading. The proton NMR spectra of a 50 mg hydrocarbon sample of a hydrocarbon stock having low olefins content (Sample B) are shown in FIGS. 5A-5D. FIGS. 5A, 5B, 5C, and 5D provide the proton NMR spectra for, respectively, the entire sample, the first (saturates) fraction, the second (olefins) fraction, and the third (unsaturated, nonolefinic) fraction. While FIGS. 5B and 5D indicate no detectable olefins, as expected, even in FIG. 5B, the NMR signal to noise ratio for olefinic hydrocarbons is low.

To improve signal to noise ratio in the proton NMR measurements, the sample loading for HPLC separation was increased from 50 mg to 160 mg. Thus, the sample aliquots were dissolved in hexane to a concentration of about 400 mg/mL, and the injection volume of 400 uL was kept constant. A representative chromatographic separation trace with 160 mg loading is shown in FIG. 6. As with the 50 mg separation trace shown in FIG. 4, the chromatographic separation trace indicates excellent separation of the fractions.

Ten samples in total with 160 mg loading of Sample A were separated and analyzed as described, with the fractions of successive runs combined. Data for these analyses is provided in Table 4. As shown, the weight percentage of olefins in the sample was consistent between runs, with a standard deviation of about 0.04% by weight for a sample containing approximately 1% olefins by weight and an average of 25 carbon atoms per hydrocarbon.

TABLE 4
Olefin Content Measurements of Samples
Containing Approx. 1% Olefins

| HPLC Results | | Proton NMR Results | | |
|---|---|---|---|---|
| | Weight | | Wt % of | |
| | Percent of | | olefins in | Wt % olefins |
| | the Olefins | | olefins | of total sample |
| | Fraction, | mol % of | fraction | (wt % of olefins |
| Sample | total recovery | olefinic | (calculation | fraction * wt % |
| Recovery | normalized | protons by | from | of olefins by |
| (Wt %) | to 100% | NMR | Formula II) | NMR) |
| 98.9 | 24.9 | 0.16 | 4.3 | 1.08 |
| 97.9 | 24.9 | 0.16 | 4.49 | 1.12 |
| 99.1 | 25.1 | 0.17 | 4.55 | 1.14 |
| 99.0 | 24.9 | 0.18 | 4.74 | 1.18 |
| 99.0 | 24.9 | 0.17 | 4.34 | 1.08 |
| 98.8 | 24.9 | 0.17 | 4.48 | 1.12 |
| 0.5 | 0.1 | 0.01 | 0.18 | 0.04 |

Protocol validation with 160 mg sample loading was performed with Sample C with a low percentage of olefins of about 0.2% by weight. Two runs with 320 mg sample loading were also performed for purpose of comparison. The fractions of either 2 runs (with 160 mg or 320 mg sample loading) or 4 runs (with 160 mg sample loading) were combined to determine the weight percentage of olefins by proton NMR. The data from these experiments is provided in Table 5.

TABLE 5
Measured Weight Percentage of Olefins in Low-Olefins Stock

| Sample Loading | Number of | Total Sample Mass | Weight Percent |
|---|---|---|---|
| per Run (mg) | Runs | for Proton NMR | Olefins (%) |
| 160 | 2 | 320 | 0.28 |
| 160 | 4 | 640 | 0.21 |
| 320 | 2 | 640 | 0.27 |

| | |
|---|---|
| Average | 0.25 |
| St. Dev | 0.04 |

As shown, the detection is accurate with a relatively low standard deviation.

The protocol was also found to be suitable for determining the weight percent of olefins in various types of samples including distillates, extracts, and raffinates.

### Additional Embodiments

Additionally or alternately, the invention can include one or more of the following embodiments.

Embodiment 1: A method for separating a hydrocarbon sample into two or more fractions, wherein at least one of the fractions is a fraction containing olefinic hydrocarbons in the hydrocarbon sample.

Embodiment 2: The method of embodiment 1, wherein at least one of the fractions is a fraction containing saturated hydrocarbons in the hydrocarbon sample.

Embodiment 3: The method of embodiment 1 or 2, wherein at least one of the fractions is a fraction containing unsaturated nonolefinic hydrocarbons.

Embodiment 4: The method of any of the preceding embodiments, wherein the method comprises contacting the hydrocarbon sample with a substrate exhibiting preferential affinity for olefinic hydrocarbons.

Embodiment 5: The method of any of the preceding embodiments, wherein the method comprises contacting the hydrocarbon sample with a substrate exhibiting preferential affinity for unsaturated nonolefinic hydrocarbons.

Embodiment 6: The method of any of the preceding embodiments, wherein the method is performed at least in part with a HPLC apparatus.

Embodiment 7: The method of embodiment 6, wherein the substrate exhibiting preferential affinity for olefinic hydrocarbons is a silver ion-loaded strong cation exchange resin.

Embodiment 8: The method of embodiment 7, wherein the olefinic hydrocarbons are eluted from the substrate with a polar solvent or polar solvent blend.

Embodiment 9: The method of embodiment 8, wherein the polar solvent comprises methylene chloride.

Embodiment 10: The method of embodiments 7-9, wherein the substrate is rinsed with a polar solvent or polar solvent blend before elution of the olefin hydrocarbons.

Embodiment 11: The method of embodiment 10, wherein the polar solvent blend comprises methylene chloride and methanol.

Embodiment 12: The method of any of embodiments 6-11, wherein the substrate exhibiting preferential affinity for unsaturated, nonolefinic hydrocarbons is a 2,4-dinitro-anilino-propyl-silica gel.

Embodiment 13: The method of any of the preceding embodiments, wherein the mass of the sample is about 50 mg or greater.

Embodiment 14: A method for quantifying olefinic hydrocarbons in a hydrocarbon sample, the method comprising measuring, by proton nuclear magnetic resonance spectroscopy, the number of signals observed at the chemical shift regions of the NMR spectrum characteristic of olefinic protons, integrating and normalizing the number of signals observed at the chemical shift regions of the NMR spectrum characteristic of olefinic protons, and calculating the weight percentage of olefinic hydrocarbons in the sample, wherein the relative number of signals observed at the chemical shift regions of the NMR spectrum characteristic of olefinic protons is not normalized to an olefinic reference signal.

Embodiment 15: A method for quantifying olefinic hydrocarbons in a hydrocarbon sample, the method comprising by proton nuclear magnetic resonance spectroscopy, the number of signals observed at the chemical shift regions of the NMR spectrum characteristic of two or more types of olefinic proton identified at Table 1 above, and quantifying the weight percentage of olefinic hydrocarbons in the hydrocarbon sample based at least in part on the number of signals observed for each of the two or more types of olefinic proton.

Embodiment 16: A kit for separation of a hydrocarbon sample into two or more fractions, wherein at least one of the fractions is a fraction containing olefinic hydrocarbons in the hydrocarbon sample.

Embodiment 17: The kit of embodiment 15, wherein the kit contains one or more of the substrates and/or solvents recited in any of embodiments 4-12.

Embodiment 18: A system for quantifying olefinic hydrocarbons in a hydrocarbon sample, the system comprising a high-pressure liquid chromatography apparatus comprising: a plurality of valves having a plurality of flow paths there between; an inlet adapted to receive a hydrocarbon sample; a solvent delivery unit; a fraction collector configured to collect one or more fractions of the hydrocarbon sample; a first column loaded with a first substrate exhibiting preferential affinity for olefinic hydrocarbons; a chromatographic trace detector; and a controller coupled to the high-pressure liquid chromatography apparatus and adapted to: direct the hydrocarbon sample, via the flow paths, to the first column to contact the first substrate; transfer the hydrocarbon sample from the first column; subsequently, to elute the olefinic hydrocarbons from the first substrate by directing a first solvent stream to the first column to contact the first substrate, to direct the eluted olefinic hydrocarbons to the detector; and subsequently to direct the eluted olefinic hydrocarbons to the fraction collector for collection as a fraction of olefinic hydrocarbons; and a proton nuclear magnetic resonance spectrometer adapted to detect chemical shift data of olefinic hydrocarbons and comprising a second controller configured to quantify the olefinic hydrocarbons in the fraction of olefinic hydrocarbons based at least in part on the chemical shift data detected for fraction of olefinic hydrocarbons.

Embodiment 19: The system of embodiment 18, wherein the high-pressure liquid chromatography apparatus further comprises a second column loaded with a second substrate exhibiting preferential affinity for unsaturated, nonolefinic hydrocarbons, and wherein the controller is further configured to: direct the hydrocarbon sample, via the flow paths, from the inlet to the second column to contact the second substrate; transfer the hydrocarbon sample from the second column to the first column; transfer the hydrocarbon sample from the first column to the detector; transfer the hydrocarbon sample from the detector to the fraction collector for collection as a first fraction; and subsequently, to elute the unsaturated, nonolefinic hydrocarbons from the second substrate by directing a second solvent stream to the second column to contact the second substrate; and, optionally, transfer the eluted unsaturated, nonolefinic hydrocarbons from the second column to the detector; and transfer the eluted unsaturated, nonolefinic hydrocarbons from the detector to the fraction collector for collection as a third fraction.

While the disclosed subject matter is described herein in terms of certain preferred embodiments, those skilled in the art will recognize that various modifications and improvements can be made to the disclosed subject matter without departing from the scope thereof. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A method for quantifying olefinic hydrocarbons in a hydrocarbon sample, the method comprising:
providing a hydrocarbon sample containing olefinic hydrocarbons;
separating olefinic hydrocarbons from the hydrocarbon sample into an olefinic fraction;
spectroscopically measuring proton resonance signals of the olefinic fraction; and
quantifying the separated olefinic hydrocarbons based at least in part on the proton resonance signals of the olefinic fraction.

2. The method of claim 1, wherein separating olefinic hydrocarbons is performed with a high-pressure liquid chromatography apparatus.

3. The method of claim 1, wherein separating the olefinic hydrocarbons comprises:
contacting the hydrocarbon sample with a substrate exhibiting preferential affinity for olefinic hydrocarbons to immobilize the olefinic hydrocarbons on the substrate exhibiting preferential affinity for olefinic hydrocarbons; and
subsequently contacting the substrate exhibiting preferential affinity for olefinic hydrocarbons with at least one polar solvent to elute the olefinic hydrocarbons from the substrate exhibiting preferential affinity for olefinic hydrocarbons to form the olefinic fraction.

4. The method of claim 3, wherein the substrate exhibiting preferential affinity for olefinic hydrocarbons is a silver ion loaded strong cation exchange resin.

5. The method of claim 4, wherein the at least one polar solvent comprises a combination of methylene chloride and methanol.

6. The method of claim 3, further comprising contacting the hydrocarbon sample with a substrate exhibiting preferential affinity for unsaturated nonolefinic hydrocarbons to immobilize the unsaturated nonolefinic hydrocarbons on the substrate exhibiting preferential affinity for unsaturated nonolefinic hydrocarbons.

7. The method of claim 6, wherein the substrate exhibiting preferential affinity for unsaturated nonolefinic hydrocarbons is a 2,4-dinitro-anilino-propyl-silica gel.

8. The method of claim 6, further comprising collecting the hydrocarbon sample as a fraction of saturated hydrocarbons after contacting the sample with the first substrate and the second substrate.

9. The method of claim 8, wherein contacting the hydrocarbon sample with the substrate exhibiting preferential affinity for unsaturated nonolefinic hydrocarbons occurs before contacting the hydrocarbon sample with the substrate exhibiting preferential affinity for olefinic hydrocarbons.

10. The method of claim 1, wherein spectroscopically measuring proton resonance signals of the olefinic fraction comprises detecting chemical shift signal data of the hydrocarbons in the olefinic fraction in a proton NMR spectrometer.

11. The method of claim 10, wherein quantifying the olefinic hydrocarbons based at least in part on the proton resonance signals of the olefinic fraction comprises correlating the chemical shift signal data of the hydrocarbons in the olefinic fraction with one or more known chemical shifts associated with olefinic hydrocarbons.

12. The method of claim 10, wherein quantifying the olefinic hydrocarbons based at least in part on the proton resonance signals of the olefinic fraction further comprises integrating the chemical shift signal data of the hydrocarbons in the olefinic fraction correlated with one or more known chemical shifts associated with olefinic hydrocarbons to generate integrated olefinic hydrocarbon data for each of the one or more chemical shifts associated with olefinic hydrocarbons.

13. The method of claim 12, wherein quantifying the olefinic hydrocarbons based at least in part on the proton resonance signals of the olefinic fraction comprises correlating the chemical shift signal data of the hydrocarbons in the olefinic fraction with a plurality of known chemical shifts associated with olefinic hydrocarbons, wherein two or more of the known chemical shifts are associated with a different subtype of olefinic hydrocarbon having a different predicted number of alkyl substitutions.

14. The method of claim 13, wherein quantifying the olefinic hydrocarbons based at least in part on the proton resonance signals of the olefinic fraction is based at least in part on the integrated olefinic hydrocarbon data for each of the two or more chemical shifts associated with a different subtype of olefinic hydrocarbon and the predicted number of alkyl substitutions for each subtype of olefinic hydrocarbon.

15. The method of claim 10, wherein quantifying the olefinic hydrocarbons based at least in part on the proton resonance signals of the olefinic fraction further comprises determining an average number of carbon atoms per hydrocarbon of the hydrocarbon sample.

16. The method of claim 15, wherein the average number of carbon atoms per hydrocarbon of the carbon sample is determined based upon the average number of carbons in the saturated hydrocarbons fraction.

17. A method for separating olefinic hydrocarbons from a hydrocarbon sample by high-pressure liquid chromatography, comprising:
providing a hydrocarbon sample;
contacting the hydrocarbon sample with a substrate exhibiting preferential affinity for unsaturated nonolefinic hydrocarbons in a first chromatography column to separate unsaturated nonolefinic hydrocarbons from the hydrocarbon sample;
transferring the hydrocarbon sample to a second chromatography column;
contacting the hydrocarbon sample with a substrate exhibiting preferential affinity for olefinic hydrocarbons in the second chromatography column to separate olefinic hydrocarbons from the hydrocarbon sample;
collecting a fraction of saturated hydrocarbons from the second chromatography column;
contacting the substrate exhibiting preferential affinity for olefinic hydrocarbons in the second chromatography column with a first at least one polar solvent to elute olefinic hydrocarbons from the second substrate; and
collecting the eluted olefinic hydrocarbons as a fraction of olefinic hydrocarbons from the second chromatography column.

18. The method of claim 17, further comprising contacting the substrate exhibiting preferential affinity for unsaturated, nonolefinic hydrocarbons in the first chromatography column with a second at least one polar solvent to elute unsaturated, nonolefinic hydrocarbons from the substrate exhibiting preferential affinity for unsaturated nonolefinic hydrocarbons; and
collecting the eluted unsaturated nonolefinic hydrocarbons as a fraction of unsaturated, nonolefinic hydrocarbons from the first chromatography column.

19. The method of claim 17, wherein the substrate exhibiting preferential affinity for unsaturated nonolefinic hydrocarbons is a 2,4-dinitro-anilino-propyl-silica gel and the substrate exhibiting preferential affinity for olefinic hydrocarbons is a silver ion loaded strong cation exchange resin.

20. The method of claim 19, wherein the first at least one polar solvent comprises a combination of methylene chloride and methanol.

21. The method of claim 19, wherein the second at least one polar solvent comprises a mixture of methylene chloride, methanol, and toluene.

22. The method of claim 17, further comprising preparing the hydrocarbon sample for separating olefinic hydrocarbons by high-pressure liquid chromatography.

23. The method of claim 22, wherein preparing the hydrocarbon sample for separating olefinic hydrocarbons by high-pressure liquid chromatography comprises dissolving the hydrocarbon sample in a nonpolar organic solvent.

24. The method of claim 20, wherein the combination of methylene chloride and methanol comprises methylene chloride in a volume percentage of about 90%.

25. A non-transitory computer readable medium comprising a set of executable instructions to direct a processor to:
obtain, from a proton nuclear magnetic resonance spectrometer, data representing a proton chemical shift signal spectrum for a fraction of olefinic hydrocarbons separated from a hydrocarbon sample,
identify, from the data representing the proton chemical shift signal spectrum, based on known chemical shifts for olefinic hydrocarbons, chemical shift signal data corresponding one or more chemical shifts characteristic of olefinic hydrocarbons;
integrate the chemical shift signal data for each of the one or more chemical shifts characteristic of an olefinic hydrocarbon; and
quantify the olefinic hydrocarbons in the hydrocarbon sample based at least in part on the integrated chemical shift signal data for each the one or more chemical shifts characteristic of an olefinic hydrocarbon.

26. The non-transitory computer readable medium of claim 25, further comprising executable instructions to direct the processor to identify and integrate, from the data representing the proton chemical shift signal spectrum, chemical shift signal data corresponding to two or more chemical shifts each characteristic of a subtype of olefinic hydrocarbon having a different predicted number of alkyl substitutions.

27. The non-transitory computer readable medium of claim 26, wherein quantifying the olefinic hydrocarbons is based at least in part on the integrated chemical shift signal data for each of the two or more chemical shifts each characteristic of a subtype of olefinic hydrocarbon and the predicted number of alkyl substitutions for each subtype of olefinic hydrocarbon.
